(19) United States
(12) Patent Application Publication
(10) Pub. No.: US 2014/0316304 A2
(43) Pub. Date: Oct. 23, 2014

(54) DEVICE AND METHOD FOR MEASURING AND ASSESSING MOBILITIES OF EXTREMITIES AND OF BODY PARTS

(71) Applicant: SportMed AG, Bitburg (DE)

(72) Inventor: Christian Egen, Bitburg (DE)

(21) Appl. No.: 13/780,537

(22) Filed: Feb. 28, 2013

(30) Foreign Application Priority Data
Feb. 28, 2012 (DE) 102012202990.7

(51) Int. Cl. A61B 5/11; A61B 5/00

(52) U.S. Cl. CPC A61B 5/11; A61B 5/0004; A61B 5/72; A61B 5/6813
USPC 600/595

(57) ABSTRACT

A device and method for measuring and diagnosing mobility of extremities and body parts of a proband, characterized by a wireless measuring device attached or fixed to an extremity or a body part to be measured of the proband, the measuring device including a sensor for three-dimensional continuous detection of position changes of the measuring device, a data processing device that wirelessly receives the measured values, an input device for manual inputs, wherein the input device is part of the wireless measuring device, an analysis program for planning a measurement and/or for a visualization of a measurement and/or for an analysis of a measurement, wherein the analysis program runs on the data processing device and computes position changes of the measuring device from the measured values of the measuring device, computes mobility of an extremity or of a body part from the position changes; and processes manually inputted inputs.

This application claims priority to German Patent Application No. 10 2012 202990.7 filed Feb. 28, 2012, this application being fully incorporated herewith by reference.

### TECHNICAL FIELD

The present invention relates to a device and a method for measuring and diagnosing mobility of extremities and of body parts of a proband, typically of a human. Mobility of extremities of a proband is measured within the diagnostics of a medic or of a physiotherapist in order to provide information about the mobility of specific joints of a patient. Then, this information can be used as a starting point of or for supervising target-oriented therapy measures.

### PRIOR ART

The physical examination of joints and body parts concerning mobility and concerning the quality of the mobility is part of the thorough examination of the muscular skeletal system and is often referred to as “manual inspection”. This inspection is divided into the quantitative measuring of the range of movement of a joint or of a body region and into the qualitative examination of the ability of movement as well as its documentation. Both aspects of the physical examination of a body region require an appropriate positioning of the patient and an isolated movement of the examined region.

During the physical examination of the quality and ability of movement, the corresponding body region is examined in terms of state, pain and muscular ability of movement, described qualitatively and documented. The qualitative detection of the muscle function is described in general by attributes on a scale ranging from immobility, through impairment up to above-average ability of movement (strength). In this regard, the assessment is based on the personal evaluation of the examiner.

Examples for such types of description can be found amongst others in
JANDA, V.: Manuelle Muskelfunktionsdiagnostik. Munich: Verlag Urban & Fischer, 4^{th} Ed., 2009.
and

KENDALL, F. P.: Muskeln-Funktionen and Tests. Munich: Verlag Elsevier Urban & Fischer, 4^{th} Ed., 2001.

The results of an examination are documented by the examiner, which leads to a permanent switch between documentation and examination on the patient. It is also common practice to dictate the results to an assistant. This possibility may be fast in execution, but at the same time it is linked to economic disadvantages and the risk of misunderstanding during the dictation.

The extremities are in particular the arms, the legs and the head of a proband or patient. With devices for measuring mobility of extremities, not only the mobility of the joints between torso and extremity, but also that of joints between two parts of an extremity, for instance mobility of an elbow or knee joint, can be measured.

Mobility of extremities of patients or probands can, for instance, be determined and analyzed according to the neutral-zero method. Starting point of a measurement according to the neutral-zero method is the so-called neutral-zero position, in which the body of the proband is in the subsequently defined starting position:
upright standing with forward-facing direction of view;
the arms hang downwards at the side of the body in a relaxed manner, with the palms of the hands facing the thighs;
the feet stand in hip width distance and parallel to each other.

The angles of the extremities to the torso and to the other parts of the extremities that result from this position are defined as zero position.

During a measurement, the examiner moves an extremity of the proband from the zero position to an end position, which refers to the position from which the extremity cannot be moved any farther in the direction of movement. Then, the angle between zero position and end position is measured.

The measurement is carried out starting from the zero position in both directions of movement, which are to be in one plane. Taking knee as an example, the maximum extension and the maximum flexion of the knee joint are measured.

According to the neutral-zero method, the measurement results are indicated in the form of a number triplet. In the example of measuring a knee joint, the following result is obtained, for instance: “5-0-110”. This result indicates that a proband is able to extend, i.e. to stretch his/her knee up to 5° out of the zero-position and to flex, i.e. to bend it up to 110°.

A continuative description of the neutral-zero method is given in the book:
MEINECKE, R.; GRÄFE, K.: Bewegungs-, Längen and Umfangsmessungen: Neutral-Null-Durchgangsmethode. Haan: Verlag Europa-Lehrmittel Nourney, Vollmer GmbH & Co. KG, 2007.

A common device for measuring mobility of extremities according to the neutral-zero method is a goniometer. A goniometer is a mechanical angle-measuring instrument with two moveable arms, between which a scale is arranged for measuring an angle. This device has the disadvantage that it comprises a great measurement uncertainty. This is caused in particular by the fact that common goniometers comprise comparatively short arms in comparison to the length of an arm or a leg of a proband, for instance. Whereas goniometers with long arms are more precise, they are substantially more difficult to handle. A further problem is the fact that it is not monitored whether the movement is carried out correctly. In addition, common goniometers do not comprise a possibility of transferring data directly to a data processing device for analysis.

This problem has for some years been solved by partially complex electro-mechanic frames for fixing and guiding the extremity of a proband in combination with data processing devices.

Thus, for instance, unexamined application DE 39 07 140 A1 shows a measurement device for the determination of the active and passive mobility of a shoulder joint. The measurement device consists of an electro-mechanical unit and a computer-controlled unit. The electro-mechanical unit is rigidly connected to the arm of the patient such that the arm may point at every solid angle possible for the shoulder joint. The electro-mechanical unit comprises two rotation axes that are coupled via a bracket, wherein the extensions of the axes meet in the center of the head of the humerus, which is thought of as a sphere.

From patent specification AT 384 544 B a method is known for the determination of the mobility of body parts by subsequent position measuring by means of electronic measurements. For this purpose, ultrasonic receivers or ultrasonic transmitters are applied to one or more body parts, which work together with assigned ultrasonic receivers or ultrasonic transmitters, which are located in fixed positions.

It is disadvantageous for such arrangements that they are very large and difficult to transport in their entirety or at least in view of the components which are in fixed positions. Thus, it is difficult to use them during physiotherapeutic home visits. Furthermore, such arrangements are very limited in their usability, since they are primarily designed for measuring one very specific motion sequence, but are not suited for being used as general measurement devices for carrying out a plurality of mobility measurements.

An angle measuring device and an angle measuring method for mobility control of muscles and/or joints responsible for the extremities of a human are known from the unexamined application DE 102 14 318 A1. In this regard, the angle measuring device comprises an electronic angle measuring sensor for the detection of an angular position of vectors that lie in a generally vertical plane, on the one hand. In this regard, the angle sensor is attached to an extremity or the torso of a proband.

It is also disadvantageous in the system that inputs before and after the measurement primarily have to be carried out via input means of the data processing devices. In order to make inputs, the examiner has to leave the proband between measurements. This is considered to be very uncomfortable and time-consuming. Furthermore, input means like keyboards etc. of data processing devices cannot be used in clinical use, since they cannot be kept sufficiently sterile.

In addition, such measuring systems based on one single sensor are very prone to measuring deviations, since there is no possibility of verifying the measurements. In addition, when repeated measurements are carried out, deviations arise due to the manifold possible variations, which are higher in most cases than the deviations which are induced by the sensor during a single measurement. Consequently, the person carrying out the treatment has to rely on the result of a single measurement, which comprises significant uncertainties.

Thus, it is the problem of the present invention to provide a device for measuring mobility of extremities and of body parts and a corresponding method for measuring mobility and states of extremities and of body parts, which comprise the following advantages:
Generating measured values of the mobility examination with a significantly reduced measurement uncertainty;
Improving handling comfort and usability for the examiner and the proband;
Promoting clinical use by improved hygiene; and
Supporting the examiner during the measurement and documentation both for single measurements and for entire complex measurement tasks.

### SUMMARY OF THE INVENTION

The above-mentioned problems are solved by a device for measuring and diagnosing mobility of extremities and of body parts according to embodiments of the present invention as well as by a method for measuring and diagnosing mobility of extremities and of body parts according to embodiments of the present invention.

In particular, the above-mentioned problems are solved by a device for measuring and diagnosing mobility of extremities and of body parts of a proband comprising a wireless measuring means that can be attached or fixed to an extremity to be measured or to a body part to be measured of the proband, wherein the measuring means comprises at least one sensor for three-dimensional continuous detection of position changes of the measuring means, a data processing device that wirelessly receives the measured values of the measuring means, an input means for manual inputs by a user, wherein the input means are part of the wireless measuring means, an analysis program for planning a measurement and/or for visualizing a measurement and/or for analyzing a measurement, wherein the analysis program runs on the data processing device and the analysis program computes position changes of the measuring means from the measured values of the measuring means, computes mobility of an extremity or of a body part from the position changes and processes inputs of the user manually inputted via the input means.

By means of the measuring device according to the invention mobility of extremities or of body parts of a proband or patient can be measured, normally by a person who examines the proband attaching the wireless measuring means to the extremity to be measured and then moving this extremity together with the measuring means in the plane to be measured. Of course, the measuring device according to the invention is also appropriate for measuring other movements of the body and of body parts. A measurement is also possible wherein the proband moves the extremity or the body part himself/herself. Herein, the wireless measuring means can be fixed to the extremity or the body part.

The at least one sensor continuously detects a three-dimensional relative movement of the wireless measuring means. In addition to three-dimensional sensors, which are able to detect a relative movement in all three space dimensions themselves, it is also possible to use a plurality of one-dimensionally or two-dimensionally measuring sensors which jointly continuously detect a three-dimensional relative movement of the wireless measuring means. By a relative movement being measured by corresponding sensors, fixed points or fixed sensor components, etc. can be omitted. Therefore, it is sufficient to use one single wireless measuring means according to the invention together with a corresponding data processing device in order to execute a precise measurement. The measuring device is thus small and handy on the whole and is also appropriate for mobile use.

As soon as at least one measurement (for example a change in altitude) is determined by at least two sensors at the same time or in parallel, it is possible to determine a mean value for this measurement. This mean value can either be computed by the measuring means itself or by the analysis program after transmission of the raw data to the data processing device. This determination of a mean value allows compensating random deviations and measurement uncertainties of the sensors.

Furthermore, before the mean value is determined, a plausibility check is carried out, which prevents falsified measured values, such as caused by irritations of the magnetic field sensor due to magnetic metal masses, for instance, from influencing the measurement.

If sensors which are based on different operating principles (acceleration, magnetic field, gravity field, position change) are furthermore used for the parallel measurement, it is also possible to compensate deviations that are caused by the measuring principles in addition to the random deviations of the sensors.

Commercial stationary computers or portable systems can be used as data processing devices, for example. Portable systems, like, for instance, notebooks, subnotebooks or tablet PCs are particularly advantageous when the measuring device is to be used for home visits to patients or probands.

A further advantage of the present invention lies in the significantly increased handling comfort for the person who examines the proband, i.e. for the examiner. The measuring means is wirelessly connected to the data processing device so that the freedom of movement of the examiner and the proband is not limited by cables etc. Preferably, the measuring means is supplied with the necessary electrical energy by an internal power source, like, for instance, a battery or an accumulator, and does then not have to be connected to an external power source.

The measuring means comprises input means for manual inputs by a user, normally the examiner or more rarely the proband himself/herself. Preferably, all functions that are necessary for a measurement of an analysis program which is executed on the data processing device can be controlled by the input means. In this way, the input means allow the examiner to stay with the proband also during an entire measurement sequence of a measurement profile. The examiner does not have to leave the proband when carrying out a measurement from a measurement sequence in order to make possible inputs on the data processing device.

It is particularly preferred to be able to carry out all inputs which are necessary for planning, execution and analysis of the measurement, and navigation in the analysis program via the input means of the wireless measuring means. Subjective or qualitative findings can particularly also be inputted. These qualitative findings can be additional findings like, for instance, the experience of pain or the ultimate feeling of the proband or further examination findings of the manual joint inspections, like, for instance, the strength of the examined musculature.

Such qualitative findings can be inputted by the user via the input means of the wireless measuring means directly during or after the quantitative measurement. Thus, the objective mobility measurement and the subjective findings are carried out by the same measuring means and in one step, which significantly simplifies and accelerates the examination of the proband.

An additional advantage lies in the increased hygiene during the measurement of mobility of extremities. The measuring means is preferably attached directly to the proband's extremity to be measured during the measurement by the examiner. Due to the wireless form, the measuring means can be designed in that it can be simply disinfected or sterilized. This can be ensured by the constructive design of the measuring means, by the selection of the used materials and by the provision of specific sealing members.

A further hygienic advantage results from the fact that the examiner can carry out all necessary inputs via the sterile measuring means itself during measuring. Thus, the examiner does not have to make inputs during measuring via non-sterile input means of the data processing device, like keyboards and the like. Due to this, a transfer of viruses and bacteria to and from the proband is avoided. Thus, the measuring device according to the invention allows measurements with significantly reduced measurement uncertainty, an optimized mobility of the measurement device in its entirety, a significantly increased handling comfort when caring for the patient and measurements with increased hygiene. By the chosen sensors, which completely do without external reference points or markers which have to be set up, advantages in view of mobility, manufacturing costs and initial efforts before the measurement are achieved.

Preferably, the sensor comprises a three-dimensional magnetic field sensor and/or a three-dimensional gyroscope and/or a three-dimensional acceleration sensor in order to detect position and orientation changes of the wireless measuring means. By means of a three-dimensional magnetic field sensor, the orientation of the measuring means corresponding to the magnetic field of the earth or to other non-natural magnetic fields can be measured in all three space dimensions. A three-dimensional gyroscope measures angular accelerations of the measuring means around all three main axes, which are caused by rotations of the measuring means. By means of the three-dimensional acceleration sensor, linear accelerations of the measuring means in all three main axes can be measured. Correspondingly, tilts of the measuring means to the gravity field of the earth are measured by the acceleration sensors.

Preferably, a measuring means according to the invention comprises a three-dimensional magnetic field sensor, a three-dimensional gyroscope as well as a three-dimensional acceleration sensor. Since all three kinds of sensors detect three-dimensional relative movements, some of the measured values or all of the measured values are at hand in an over-determined manner. An orientation vector is computed from these measured values of the different sensor types by means of so-called “strapdown calculation” and by means of a so-called “Kalman filter” and the position and orientation change of the measuring means are then computed from this. This calculation can already take place in the measuring means itself or in the analysis program or in parts in both components.

Since the measured values of the position changes are over-determined, random deviations of the sensors and other measurement failures like completely non-plausible measured values, can be compensated, which leads to a very precise position and orientation determination of the measuring means relative to the starting position on the whole.

Preferably, a plurality of measuring means is wirelessly connected to the data processing device. The data processing device is able to receive and to process the data of a plurality of measuring means, like, for instance, of four measuring means. Thus, it is possible to examine twin movements of the proband. In this regard, every measuring means can unambiguously identify itself to the data processing device and the analysis program so that the received measured values can be unambiguously assigned to the corresponding measuring means. Such a measuring means can, for instance, be used in order to develop and to use new methods for mobility and posture measurements—which exceed the neutral-zero method.

Preferably, the input means are designed for multi-dimensional inputs, in particular for navigation within the analysis program or for inputting findings. Such input means for multi-dimensional inputs may be designed as a—directional pad, for instance, an arrangement of four direction pointers and a confirm button or as a touch sensitive surface for the interaction of the examiner with the measuring means.

Preferably, the sensor detects the tilt of the measuring means and the analysis program computes manual input values of the user from the tilt. Thus, the user can, for instance, input values for the experience of pain during a movement on a scale of 1 to 10, for instance, by simply and intuitively tilting the measuring means.

Preferably, the sensors do not need any reference points in the measuring field. Reference points are fixed points in space which are necessary in the prior art for the determination of an absolute position of the measuring means. In a preferred embodiment, the present measuring means do not need corresponding fixed points, since it is not necessary to determine an absolute position of the measuring means. Instead, the relative position and optionally the relative orientation of the measuring means in view of a starting point are used. For the analysis of mobility of extremities—like, for instance, according to a neutral-zero method—the zero position of the extremity is the relative starting point. This relative measurement has the advantage that no additional components, like, for instance, signal sources which are fixed in space or sensors which are fixed in space have to be set up and calibrated for carrying out a measurement. This is particularly advantageous during mobile use.

Preferably, the manual inputs which are possible at the wireless measuring means by means of the input means, comprise identifying a zero position of the extremity and/or of an end position of the extremity and/or noting a subjective finding concerning the proband during the measurement. By means of the above-mentioned inputs, it is possible to carry out essential inputs which have to be carried out during a measurement directly via the input means of the measuring means. Thus, the examiner can stay with the proband during the execution of the measurement and does not have to switch back and forth between the proband and the data processing device. Furthermore, it is ensured that the subjective findings are assigned to the correct measurement, i.e. to the correct joint.

Preferably, the analysis program comprises a teach-in module for calibration of the measurement plane before the real measurement is executed. For the supervision of the correct movement sequence of the extremity of the proband it can be necessary to teach the correct movement sequence to the measuring device. The teach in is particularly not used for sensory calibration, but the determination of the plane which is relevant for the measurement, i.e. the plane in which the movement is to be measured. During teach in, the measurement device determines the plane of the subsequent measurement from the defined executed movement sequence. The teach-in module instructs the examiner to comply with the correct movement sequence.

Preferably, the analysis program comprises a pool of pre-defined single measurements, which can be combined into a measurement profile by the analysis program. In this regard, during preparation of the measurement, the analysis program provides a plurality of pre-defined single measurements, which are combined in a pool. From this pool, which particularly contains all standard-single measurements of the neutral-zero method, the examiner chooses the single measurements to be executed, and arranges them in the analysis program to an individual measurement profile for this specific proband. Herewith, the real measurements on the proband can be carried out uninterruptedly one after the other. In addition, recurring measurement profiles can be saved.

In the context of the measurements, information about the joint to be examined, the movement direction to be examined, information about the specific neutral-zero position of the joint to be examined, information about a teach-in measure to be executed, requirements of the analysis of the measurement, graphic illustrations for the visualization of the movement sequence and pre-defined input options for additional subjective findings can be displayed to the examiner by the analysis program. During the actual measurement, the analysis program may display a graphic illustration of the extremity, which moves simultaneously to the movement of the real extremity, in order to signalize the measurement progress and a successful measurement to the examiner.

Furthermore, the problem according to the invention is solved by a method for measuring and diagnosing mobility of extremities and of body parts of a proband, comprising the following steps:
a. providing a wireless measuring means that can be attached or fixed to an extremity to be measured or a body part to be measured of the proband, wherein the measuring means comprises sensors for the three-dimensional continuous detection of position changes of the measuring means;
b. providing a data processing device which wirelessly receives the measured values of the measuring means;
c. providing an input means for manual input by a user, wherein the input means is part of the wireless measuring means;
d. attaching or fixing the measuring means to the extremity to be measured or the body part to be measured of the proband;
e. moving the extremity of the proband;
f. manually inputting a subjective finding of the proband via the input means; and
g. analyzing the measurement by the analysis program.

Thus, for the reasons which were already explained above with regard to the measuring device, the method according to the invention also allows measurements with significantly reduced measurement uncertainty, an optimized mobility of the entire measuring device, significantly increased handling comfort and an improved care for the patient and measurements with improved hygiene. Moreover, by the measurement of position changes of the measuring means complex fixed points or stationary reference points of the sensors are omitted, which makes the measuring device significantly cheaper on the whole. Preferably, a measuring device according to patent claim 1 is used for carrying out the method.

In particular, subjective findings can now be inputted directly via the wireless measuring means and can thus be documented. The objective mobility measurement and the subjective finding can be executed by the same measuring means and in one step, which significantly simplifies and accelerates the examination of the proband.

Preferably, the method moreover comprises a step of choosing the measurement to be carried out in an analysis program out of a pool of pre-defined single measurements. The examiner can select single measurements to be executed from the pool in advance and combine these to form an individual measurement profile for the proband. Due to this, the efficiency during the actual measurement of the proband can be increased by it being possible for the individual measurements to be offered and carried out by the analysis program one after the other.

Preferably, the user can navigate in the analysis program by means of the input means of the wireless measuring means or can choose the measurements to be executed. The examiner is able to make inputs for the analysis program directly on the wireless measuring means, without having to leave the proband during the execution of the measurement.

Preferably, the method furthermore comprises a teach-in step, by which the movement plane to be measured is recognized and the analysis program is calibrated. With the teach-in step, the plane which is foreseen for the measurement is taught to the analysis program by defined movement in the plane.

Preferably, the analysis step comprises a partial step of a continuous computing of the relative position and relative orientation in space of the measuring means out of a plurality of over-determined measured values of three-dimensional sensors. Herewith, in particular the end position and the end orientation of the extremity are computed. By the computing of the end position and the end orientation on the basis of over-determined measured values, the measurement precision is very high despite measuring without reference points with fixed locations. The starting and end positions of the extremity or of the body part, on the other hand, can be determined by a manual input at the measuring means or they can be derived after the complete movement directly from the extremes of the continuous measurement.

Preferably, the method furthermore comprises a step of numeric and/or graphic real-time output of the measuring results during the measurement on a screen of the data processing device. Due to this, the examiner can see in real time on a screen of the data processing device how the extremity of the proband moves during the measurement and whether the measurement is taking place successfully. If the extremity leaves the pre-determined measurement plane on the way from the zero position to an end position, the analysis program is able to output a warning. Thus, it is possible to correct a movement which was not carried out correctly immediately and to ensure a correct measurement result.

### SHORT DESCRIPTION OF THE DRAWINGS

In the following, preferred embodiments of the present invention are described in the context of the figures. Therein, the following is shown:

FIG. 1 a block diagram of components of an embodiment of a device for measuring mobility of extremities according to the invention;

FIG. 2 a three-dimensional view of an embodiment of a measuring means according the invention for measuring mobility of extremities;

FIG. 3 a three-dimensional exploded view of the embodiment of a measuring means for measuring mobility of extremities according to the invention according to FIG. 2

FIG. 4 a schematic overview of the device for measuring mobility of extremities according to the invention during a measurement;

FIG. 5 a screenshot of an embodiment of the analysis program according to the invention while an individual measurement profile is combined from a pool of single measurements;

FIG. 6 a screenshot of an embodiment of the analysis program according to the invention while the zero position of an elbow joint of the proband is defined;

FIG. 7 a screenshot of an embodiment of the analysis program according to the invention during the execution of a measurement of the mobility of the elbow joint of the proband;

FIG. 8 a screenshot of an embodiment of the analysis program according to the invention, during the output of the measured results of the measurement of a mobility of a hip in the in the form of documentation according to the neutral-zero method; and

FIG. 9 a screenshot of an embodiment of an analysis program according to the invention, during the input of subjective findings during the measurement.

### DETAILED DESCRIPTION OF PREFERRED EMBODIMENTS

In the following, preferred embodiments of the present invention are described in detail with reference to the figures. Individual features of specific embodiments can be combined with features of other embodiments when it is reasonable.

The device for measuring as well as the corresponding method are described in the following in a preferred situation in which a proband is examined by an examiner, by the examiner attaching a measuring means to an extremity of the proband and then guiding the extremity of the proband to the desired movement plane. Alternatively, however, it is also possible for the movements to be carried out by the proband alone.

Furthermore, the device and the method are explained using the example of the neutral-zero method. Another suitable method for the determination of mobility of extremities can be also supported.

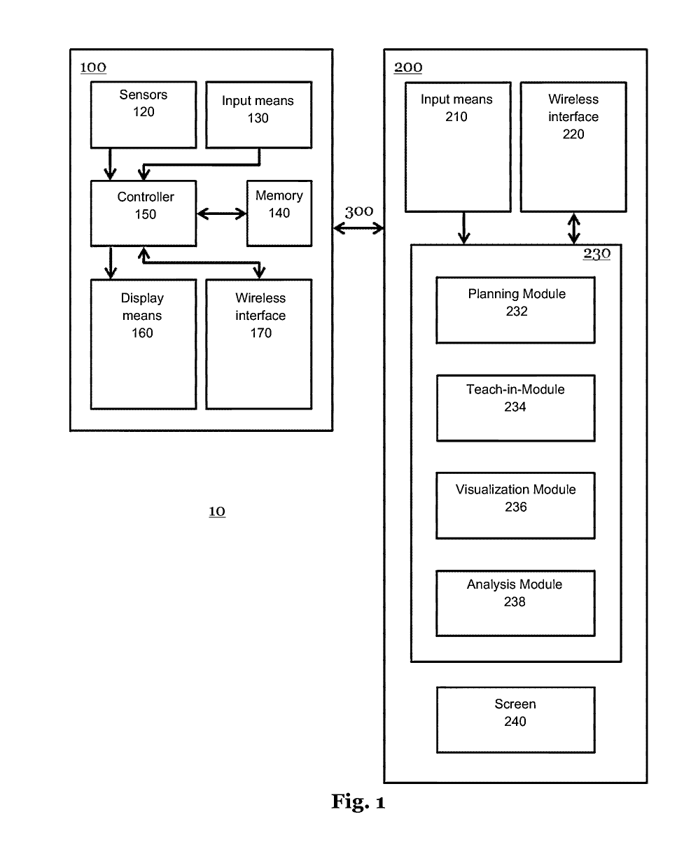

FIG. 1 shows a block diagram of the components of an embodiment of a device for measuring mobility of extremities 10 according to the invention, which is also referred to as measuring device 10 in the following. As its main components, the measuring device 10 comprises a wireless measuring means 100, which is attached to the extremity for measurement, and a data processing device 200 for analysis and documentation. The measuring means 100 and the data processing device 200 are connected to each other in data matters via a radio link 300. Radio link 300 is preferably bi-directionally designed. This means that data can be transmitted from the measuring means 100 to the data processing device 200 and vice versa. A unidirectional radio link 300 is also possible, which transmits the measured values from the measuring means 100 to the data processing device 200. As radio standard, for instance Bluetooth, W-LAN or another digital standard can be used.

The sensor data is transmitted wirelessly to the data processing device 200. In the analysis program 230, which is executed there, the analysis of the detected measured values is carried out, wherein relative movements and relative orientations of the extremity to be measured are calculated from the measured values. The mobility of the extremity can then be computed from this for instance by the use of the neutral-zero method and it can be displayed. The analysis can be outputted numerically, graphically or audio-visually via the output means (screen or printer) of the data processing device or via output means of the wireless measuring means.

The measuring means 100 commonly comprises several sensors 120 for the detection of orientation and position. The sensors 120 preferably comprise a three-dimensional magnetic field sensor 122, a three-dimensional gyroscope 124 and a three-dimensional acceleration sensor 126. Other sensors, like, for instance, gravity field sensors, can also be used. The sensors preferably determine relative orientation and position changes, with the measurements being partially determined in an over-determined manner. Thus, the relative orientation and position changes of the measuring means can be computed very precisely.

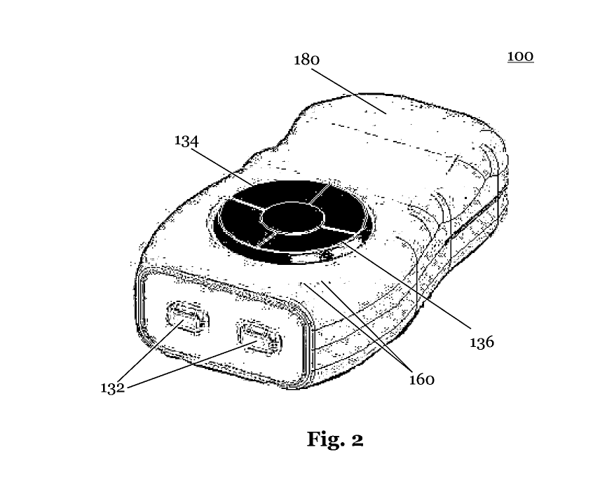

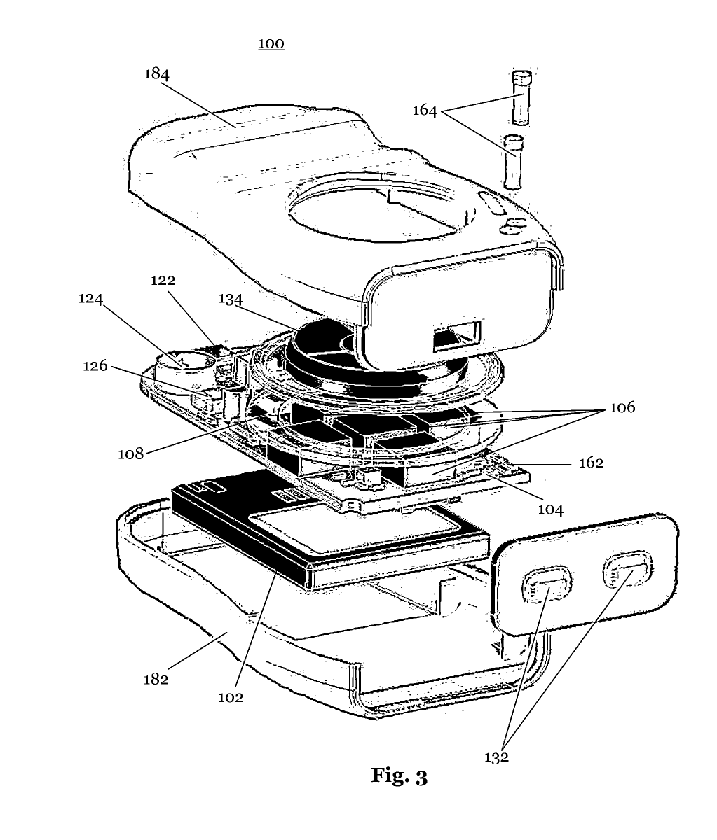

The measuring means 100 furthermore comprises input means 130, by which it is possible to carry out manual inputs concerning the measurements and to control the analysis program 230. The input means 130 can comprise all kinds of buttons and sensors which are suitable for converting a manual input into an electrical signal that can be analyzed by the measuring means 100 itself or by the data processing device 200. As it is shown in FIGS. 2 and 3, the measuring device 100 comprises, on the side which is opposed to the proband, a plurality of directional pad push buttons 134, which form a directional pad together. Herein, the directional pad push buttons 134 traditionally represent the directions “up”, “down”, “left” and “right”. Other orientations are also possible. Additionally, the directional pad push buttons comprise a central push button 136, which can be used for instance for the confirmation of a choice, which is done via the directional pad. The push button 136 is also preferably used for the definition of a zero position and a position of the measurement.

The signals which are outputted by the sensors 120 and the input means 130 are supplied to a controller 150. In this regard, the signals of the sensors 120 can be analog or digital signals. If necessary, the controller converts the signals from an analog to a digital signal, stores these signals in a memory 140, where appropriate, carries out interim calculations and transmits the measured values to the data processing device 200 by means of a wireless interface 170 via the radio link 300.

The measuring means 100 can also comprise indication means 160 in order to suggest specific operation conditions (for instance empty accumulator 120) or the correct connection via the radio link 300. The indication means may also serve to give an optical feedback to the user of inputs which are made. In addition, the measuring means can acknowledge every input with an acoustic signal. The indication means 160 may comprise luminous diodes 162, LCD displays or screens of all kinds (not shown).

The measuring means 100 can furthermore comprise fixation means, by which it is fixed to the extremity 22 to be measured of a proband 20. Such fixation means may possibly comprise a hook-and-loop tape or a belt.

The data processing device 200 preferably comprises a commercial computer or a commercial portable notebook. The data processing device 200 comprises common input means 210, like, for instance, a keyboard, a computer mouse or a touch-sensitive surface of a screen. Among other things, it is possible to control the analysis program 230 via these input means 210.

The analysis program 230 comprises an analysis module 238 that computes position and orientation changes of the measuring means 100 from the measured values of the measuring means 100 and deduces a mobility of an extremity from this. In addition to the measured values, the analysis program also receives and computes the manual inputs of the user via the input means 130, 210.

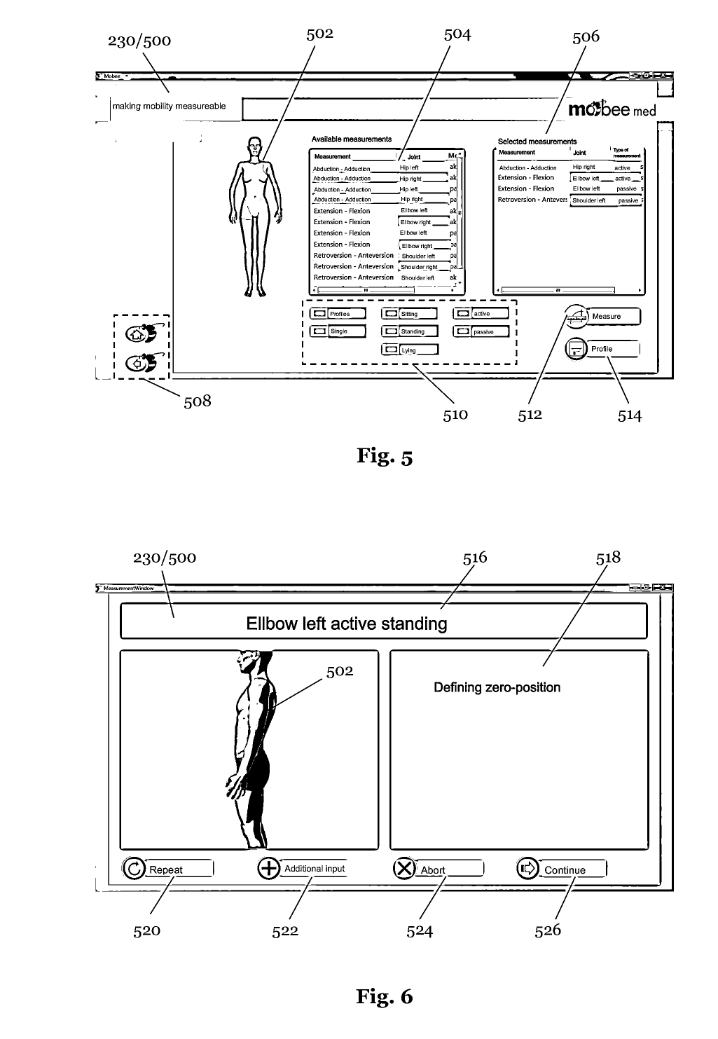

Furthermore, the analysis program 230 comprises a planning module 232 that serves the purpose of the preparation of the measurements to be executed. The preparation can consist of a definition of one or more single measurements or of a selection of single measurements out of a pool 504 of single measurements. These can be combined into an individual measurement profile 506 for the respective proband, as shown in FIG. 5. These measurement profiles 506 can be also stored and reused later on.

Furthermore, the analysis program 230 comprises a teach-in module 234 that serves the purpose of defining the measurement plane in which the measurement is to take place subsequently. Thus, in succession, the movement sequence can be supervised during the execution of the measurement and possible mistakes can be signalized directly to the examiner.

A visualization module 236 of the analysis program 230 supports the proband 20 and the examiner during the execution of the measurement by graphically screening the movement to be carried out before the measurement. Furthermore, the data which is transmitted by the measuring means 100 during the measurement is analyzed by the analysis module 238 in real time and the current position of the extremity is visualized online in the analysis program 230.

An analysis module 238 of the analysis program 230 receives the raw data that is transmitted from the measuring means 100 and computes the current relative position and relative orientation of the measuring means 100 from this. This relative position and relative orientation is used by the visualization module 236 in order to display in real time the current position and orientation of the extremity, on the one hand. Furthermore, after the completion of the measurement, the analysis module 238 computes the mobility of the extremities of the proband in the form of the desired representative indicators, in particular as number triplets 534 according to the above described neutral-zero method.

A screen 240 of the data processing device 200 serves the purpose of the graphic display of the outputs of the analysis program 230.

FIGS. 2 and 3 show a three-dimensional view of an embodiment of a measuring means 100 according to the invention for measuring mobility of extremities, with FIG. 2 showing a complete view and FIG. 3 an exploded view.

The measuring means 100 comprises a housing 180, consisting of a lower shell 182 and an upper shell 184. The housing 180 protects the components arranged inside and serves the purpose of the simple cleaning and disinfection of the part of the measuring device 100 which gets in contact with the proband.

The manual input means 130 pass through the upper shell 184 of the housing 180. The shown input means 130 comprise a front push button 132, which can be activated by the examiner even when the measuring means 100 is attached and fixed by a hand of the examiner to the extremity 22 of the proband 20. Furthermore, the input means comprise a directional pad, consisting of directional pad push buttons 134 and a central push button 136.

The manual input means 130 serve the purpose of the hygienic and comfortable control of the analysis program 230 and of the actual measurement. If aspects of hygiene and comfort are less relevant, the analysis program 230 can also be controlled via the common input means 210 of a data processing device 200, like, for instance, via a keyboard, a mouse or a touch screen during the preparation and the analysis of the measurement.

The measuring means 100 in the embodiment which is shown in FIGS. 2 and 3 also comprises indication means 160, which consist of luminous diodes 162 on a control board 104 and corresponding optical wave guides 164. These indication means are used as explained above in order to indicate specific conditions of the measuring means or to confirm manual inputs optically.

Besides the luminous diodes 162, the control board 104 furthermore comprises a three-dimensional magnetic field sensor 122, a three-dimensional gyroscope 124 and a three-dimensional acceleration sensor 126, which, as explained above, provide corresponding measured values for the determination of the relative orientation and position. Additionally, a gravity field sensor can also be arranged on the control board 104.

Furthermore, on the control board a plurality of push-button sensors 106 is arranged, which converts the pressure impulses of the directional pad push buttons 134 and of the central push button 136 into electric signals. The control board 104 is the central element of the measuring means 100 and comprises moreover a controller 150, a memory 140 and a wireless interface 170, which is not shown in FIG. 3.

The measuring means 100 is protected against the intrusion of liquids or solid dirt particles via seals like, for instance, a sealing ring 108. Besides the protection of the electronics this particularly serves the purpose of the simplified cleaning and disinfection of the measuring means 100 after direct contact with the proband 20.

Furthermore, an energy source 102, like, for instance, an accumulator or a battery is stored in the housing 180, which supplies the measuring means 100 with electric energy during the operation.

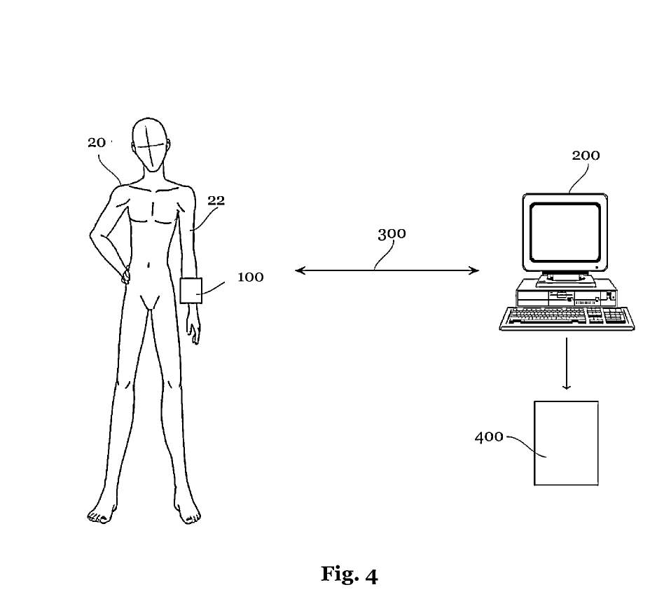

FIG. 4 shows a schematic overview of the interaction of a proband 20 with an embodiment of the device 10 for measuring mobility of extremities according to the invention. In this regard, the measuring means 100 is fixed to an extremity 22—here to the forearm—of the proband 20. The measuring means 100 stays in contact with the data processing device 200 via the radio link 300.

In the data processing device 200, the analysis program 230 is executed, which can be operated in particular by the input means 130 of the measuring means 100.

The results of a measurement are preferably outputted as measurement record 400. Its output can be done via a display means 240 of the data processing device 200, here a screen, or as a print via a linked printer (not shown) and can also be digitally stored.

FIGS. 5-9 show screenshots of a user interface 500 of the analysis program 230 in the different phases “preparation”, “execution” and “analysis” of a measurement of mobility of extremities. The individual functions and the use of the analysis program 230 are explained by means of these figures in the following.

As an initial situation, a proband 20 is to be examined by an examiner. The analysis program 230 is installed on the data processing device 200 and has been started. The user interface 500 of the analysis program 230 is shown on the screen 240 of the data processing device 200. The measuring means 100 is wirelessly connected to the data processing device 200 via the radio link 300.

The examiner controls the analysis program 230 via the input means 130 of the measuring means 100. The analysis program 230 with its user interface 500 is designed in that all inputs which are necessary for planning, execution and analysis of the measurement can be done easily and rapidly via the measuring means 100. Alternatively, the inputs can be also done via the common input means 210 of the data processing device 200.

FIG. 5 shows the analysis program 230 during the preparation of the measurement, in the step of combining an individual measurement profile 506 out of a pool 504 of single measurements. The single measurements are selected via the directional pad 134, 136 of the measuring means 100 and are combined to form an individual measurement profile 506. In order to facilitate the selection, the single measurements of the pool 504 can be presorted by specific filters 510. Possible categories for such filters 510 are “profile” and/or “single” and “sitting” and/or “standing” and/or “lying” and “active” and/or “passive”.

The user is guided through the analysis program 230 via navigation helps 508. They allow, for instance, the return to the starting page by means of a so-called “home-button” or one step back in the program sequence to be taken.

After the profile combination has been completed, the measurement is started via the field measurement start 512. The profile 506 can also be stored before the execution of the measurement via the profile management 514. It is also possible via the profile management 514 to load a profile 506 which is already stored instead of defining a new one. Furthermore, it is possible to modify a loaded profile 506 before the measurement is executed, if this is necessary.

FIG. 6 shows a further step of the preparation of a measurement, namely the definition of the zero position. As already explained above, a measurement according to the neutral-zero method always starts from a predefined initial position, the so-called zero position. This principle of the predefined initial position is also transferred, in the course of the measurement method according to the invention, to the definition of a starting point of a relative coordinate system of the measuring device 10. Since the detected measured values are—as explained above—mostly relative values, the end position and the end orientation of an extremity can be computed via the defined zero position.

The user interface 500 of the analysis program 230 indicates the current measurement 516 and explains which steps have to be done next by means of operation instructions 518. In addition, the analysis program 230 offers the possibility of repeating a measurement via a button 520, of inputting additional inputs 522, like, for instance, the experience of pain or the ultimate feeling of the proband, of aborting the measurement 524 or of proceeding with the next step of the measurement by means of the button 526.

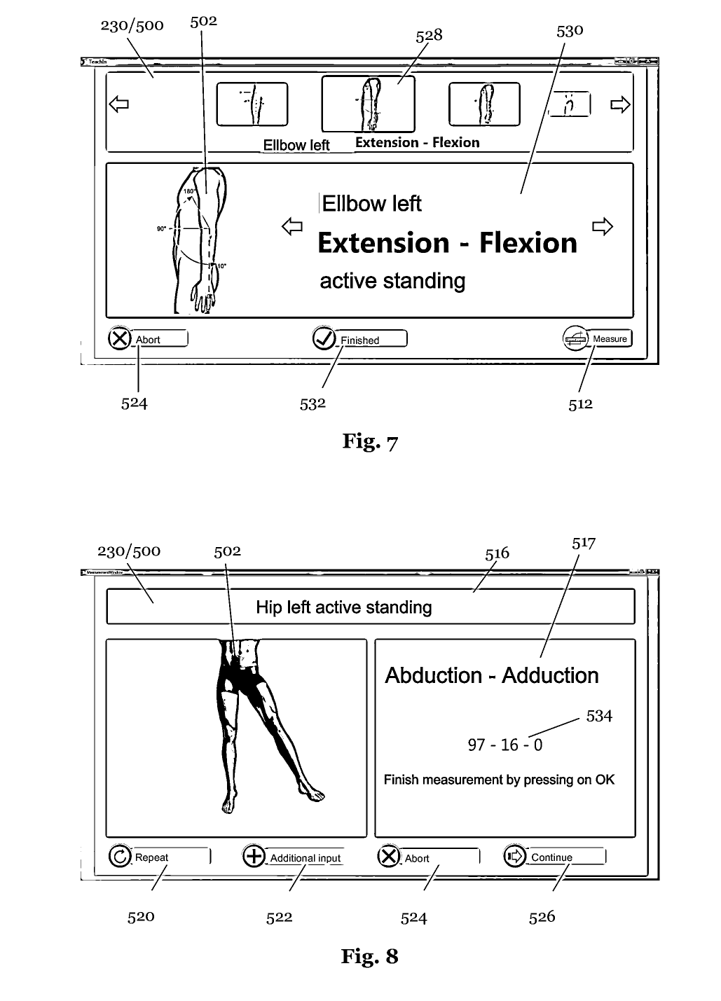

FIG. 7 shows the user interface 500 of the analysis program 230 during the execution of a measurement. A graphic representation 502 of the movement to be carried out facilitates the intuitive understanding of the measurement mentioned in the title 530. Above the window with the representation 502 and the title 530, a navigation bar 528 is arranged, by which the user can switch by means of the arrows left and right between the individual measurements of the measurement profile 506. Furthermore, this representation gives a survey of past and future measurements. During the measurement, the graphic representation 502 preferably shows the current position and orientation of the extremity 22 to be measured of the proband 20 in real time.

In addition, this real-time representation 502 is used in order to detect the movement sequence of the extremity to be measured 22 and to indicate deviations out of the measurement plane visually and/or acoustically, if applicable.

FIG. 8 shows the user interface 500 of the analysis program 230 after the analysis of a measurement. Besides the title 516 and the declaration of the current measurement 517, a graphic representation 502 of the movement carried out just is shown. Preferably, the graphic representation 502 shows the movement of the extremity 22 of the proband 20, which was detected during the measurement. In this preferred embodiment, the actual measurement result is displayed in the form of a number triplet 534, according to the neutral-zero method.

Additionally, in the measurement window, the analysis program 230 also offers the possibility of repeating a measurement by means of button 520, of documenting additional inputs such as pain and/or ultimate feeling by means of button 522, of aborting the measurement by means of the button 524 or of proceeding with the next step by means of button 526.

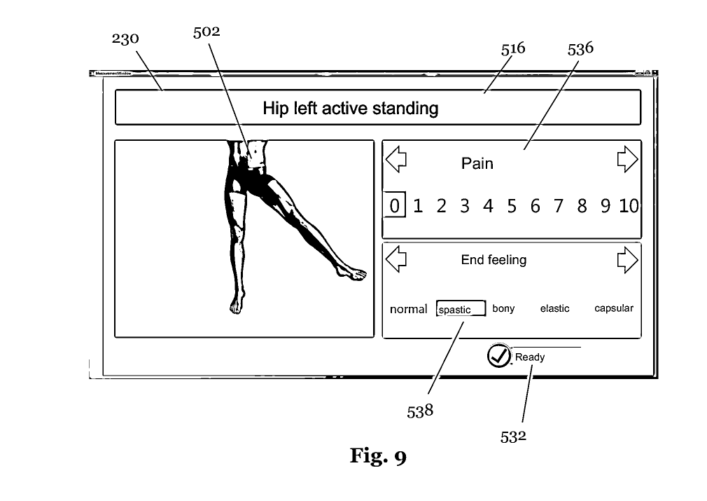

FIG. 9 shows the user interface 500 of the analysis program 230 during the input of such additional inputs, here “pain” (scale 536) and “ultimate feeling” (scale 538). After the measurement, the user can directly move the selection cursor, i.e. a graphically highlighted field, to the left and to the right via the input means 130 of the measuring means 100 and can then select the desired finding.

After the inputs made have been confirmed via the done button 532, this single measurement is completed and it is possible to proceed to the next single measurement of the measurement profile 506. After the last measurement of a measurement profile 506, the analysis program 230 offers several possibilities of data analysis and data storage.

### LIST OF REFERENCE SIGNS

10 device for measuring and diagnosing mobility of extremities and of body parts
20 proband
22 extremity
100 measuring means
102 energy source
104 control board
106 push-button sensors
108 sealing rings
110 mounting means
120 sensors
122 three-dimensional magnetic field sensor
124 three-dimensional gyroscope
126 three-dimensional acceleration sensor
130 input means
132 front push button
134 directional pad push buttons
136 central push button
140 memory
150 controller
160 indication means
162 luminous diodes
164 optical waveguide
170 wireless interface
180 housing
182 lower shell
184 upper shell
200 data processing device
210 input means
220 wireless interface
230 analysis program
232 planning module
234 teach-in module
236 visualization module
238 analysis module
240 screen
300 radio link
400 measurement record
500 user interface of the analysis program 230
502 graphic representation
504 list of possible single measurements/pool
506 measurement profile
508 navigation helps
510 filter
512 measurement start button
514 profile management
516 title of a single measurement
517 designation of a single measurement
518 operating instruction
520 repeated measurement button
522 additional input button
524 measurement abort button
526 measurement continuation button
528 navigation bar
530 single-measurement title
532 done button
534 measurement result indication
536 subjective finding pain
538 subjective finding ultimate feeling

1. A device for measuring and diagnosing mobility of extremities and of body parts of a proband, comprising:
a. a wireless measuring means that can be attached or fixed to an extremity to be measured or to a body part to be measured of the proband, wherein the measuring means comprises at least one sensor for three-dimensional continuous detection of position changes of the measuring means;
b. a data processing device, that wirelessly receives the measured values of the measuring means;
c. an input means for manual inputs by a user, wherein the input means are part of the wireless measuring means;
d. an analysis program for planning a measurement and/or for a visualization of a measurement and/or for an analysis of a measurement; wherein
e. the analysis program runs on the data processing device and the analysis program computes position changes of the measuring means from the measured values of the measuring means;
f. computes mobility of an extremity or of a body part from the position changes; and
g. processes inputs of the user manually inputted via the input means.

2. A device according to claim 1, wherein the sensor comprises a three-dimensional magnetic field sensor and/or a three-dimensional gyroscope and/or a three-dimensional acceleration sensor in order to detect position and orientation changes of the wireless measuring means

3. A device according to claim 1, wherein the analysis program determines all three space coordinates and the position of the measuring means from the position changes.

4. A device according to claim 1, wherein the measured values of the sensors of the position and orientation changes are over-determined.

5. A device according to claim 1, wherein the input means are designed for multi-dimensional inputs, in particular for the navigation in the analysis program or for inputting findings.

6. A device according to claim 1, wherein the sensor detects the tilts of the measuring means and the analysis program computes manual input values of the user from the tilt.

7. A device according to claim 1, wherein the sensors do not need any reference points in the measuring field.

8. A device according to claim 1, wherein the manual inputs comprise identifying a zero-position of the extremity and/or of an end position of the extremity and/or noting a subjective finding concerning the proband during the measurement.

9. A device according to claim 1, wherein the analysis program comprises a teach-in module for calibration of the measurement plane before the real measurement is executed.

10. A device according to claim 1, wherein the analysis program comprises a pool of pre-defined single measurements, which can be combined into a measurement profile by the analysis program.

11. A method for measuring and diagnosing mobility of extremities and of body parts of a proband, comprising the following steps:
a. providing a wireless measuring means that can be attached or fixed to an extremity to be measured or to a body part to be measured of the proband, wherein the measuring means comprises sensors for the three-dimensional continuous detection of position changes of the measuring means;
b. providing a data processing device, which wirelessly receives the measured values of the measuring means;
c. providing an input means for manual input by a user, wherein the input means is part of the wireless measuring means;
d. attaching or fixing the measuring means to the extremity to be measured or to the body part to be measured of the proband;
e. moving the extremity of the proband;
f. manually inputting a subjective finding of the proband via the input means; and
g. analyzing the measurement by the analysis program.

12. A method according to claim 11, further comprising a step of choosing the measurement to be carried out in an analysis program out of a pool of pre-defined single measurements.

13. A method according to claim 11, wherein the user can navigate in the analysis program by means of the input means of the wireless measuring means or can choose the measurements to be executed.

14. A method according to claim 11, further comprising a teach-in step, by which the movement plane to be measured is recognized and the analysis program is calibrated.

15. A method according to claim 11, wherein the analysis step comprises a partial step of a continuous computing of the relative position and orientation in space out of a plurality of over-determined measured results of three-dimensional sensors.

16. A method according to claim 11, further comprising a step of numeric and/or graphic real-time output of the measuring results during the measurement on a screen of the data processing device.
